(19) United States
(12) Patent Application Publication
(10) Pub. No.: US 2013/0231605 A1
(43) Pub. Date: Sep. 5, 2013

(54) MULTI-PURPOSE ASPIRATION/IRRIGATION/POLISHING TIPS SUITABLE FOR CATARACT SURGERIES AND RELATED METHODS

(71) Applicant: WAKE FOREST UNIVERSITY HEALTH SCIENCES, Winston-Salem, NC (US)

(72) Inventor: Keith Andrew Walter, Lewisville, NC (US)

(73) Assignee: Wake Forest University Health Sciences, Winston-Salem, NC (US)

(21) Appl. No.: 13/783,775

(22) Filed: Mar. 4, 2013

Related U.S. Application Data
(60) Provisional application No. 61/606,648, filed on Mar. 5, 2012.

(51) Int. Cl. A61M 3/02; A61F 9/008; A61F 9/007

(52) U.S. Cl. CPC A61M 3/0283; A61F 9/00745; A61F 9/008
USPC 604/22; 604/27; 604/43

(57) ABSTRACT

Devices and methods for cataract surgery include a tip with a textured outer surface that is configured to gently scrape the capsule bag to allow aspiration of cortical tissue.

### RELATED APPLICATIONS

This application claims the benefit of and priority to U.S. Provisional Application Ser. No. 61/606,648, filed Mar. 5, 2012, the contents of which are hereby incorporated by reference as if recited in full herein.

### FIELD OF THE INVENTION

This invention relates to aspiration tips that are particularly suitable for use in ophthalmic surgery such as, for example, phacoemulsification including ultrasonic and femtosecond laser cataract surgery.

### BACKGROUND OF THE INVENTION

In the United States, the majority of cataract lenses are removed by a surgical procedure known as phacoemulsification. During this procedure, a cutting tip is inserted into the diseased lens and vibrated ultrasonically. The vibrating cutting tip liquefies or emulsifies the lens so that the lens can be aspirated out of the eye. The diseased lens, once removed, is then typically replaced by an artificial lens.

More recently, femtosecond lasers have been proposed for use in cataract surgeries. The femtosecond laser has the capability to assist the fragmentation (laser phacoemulsification or breaking up) of the cataract. Generally stated, the laser applies a number of pulses to the lens in a pre-designed pattern which then allows the surgeon to remove the lens matter. See, e.g., Nagy et al, Initial clinical evaluation of an intraocular femtosecond laser in cataract surgery. J Refract Surg 2009; 25:1053-60.

Prior to inserting the artificial lens, softer or attached cortical material that was not removed during the initial step is aspirated from the eye. Typically, this is done using a tip that is similar to the ultrasound phacoemulsification tip, but with a smaller opening at the distal end and without the ultrasonic vibration. The aspiration tip can also be used to polish the posterior capsule to remove residual cortical fibers or epithelium cells to reduce the risk of posterior capsule opacification or other undesired events. Conventional aspiration tips have been made from titanium or stainless steel with highly polished surfaces to reduce burrs or sharp edges. Other aspiration tips use silicone rubber tip caps that reside over the metal tips. See, U.S. Pat. No. 5,718,677. More recently, dual function aspiration tips such as the MicroSmooth® sleeve from Alcon, Inc., that can both irrigate and aspirate have been used. See also, U.S. Pat. No. 7,967,775. The contents of these patent documents are hereby incorporated by reference as if recited in full herein.

Despite the above tips, often a J-shaped cannula or other tool must be inserted into the capsule bag during capsule polishing to help detach cortical material that is resistant to aspiration using just the aspiration and irrigation tip. Thus, there remains a need for tips that can facilitate cortical clean-up and/or polishing of the capsule bag to prevent posterior capsular opacification.

### Summary of Embodiments of the Invention

Embodiments of the invention are directed to providing a multipurpose tool tip that can be used during cataract surgeries.

Embodiments of the invention provide surgical tools suitable for polishing of a capsule bag during ophthalmic cataract surgery.

Embodiments of the invention provide surgical tools suitable for facilitating the dismantling or aspiration of a lens during laser-phaco surgery, such as during or after femtosecond laser treatment for cataract surgery to remove nuclear fragments and/or epinucleus.

Some aspects are directed to methods of performing cataract surgery. The methods include: (a) performing a phacoemulsification procedure on an eye of a patient; then (b) inserting, in vivo, an elastomeric tip of an aspiration/irrigation tool having a textured patch on an outer surface thereof into a capsule bag of a patient; then (c) manually moving the tip to cause the textured surface to contact cortical tissue; and then (d) aspirating cortical tissue using the tip.

The tip can have a non-textured smooth outer surface proximate the irrigation/aspiration port and the smooth outer surface can cover a greater surface area than a surface area of the textured patch.

The textured patch can reside only on a distalmost end of the tip.

The textured patch can cover only a rounded distal end of the tip a distance forward of the aspiration portion.

The tip can have a smooth surface opposite the textured patch, the method comprising rotating the tip so that the textured surface faces the cortical tissue after the inserting step.

Other embodiments are directed to multi-purpose irrigation/aspiration tips for use in combination with a surgical system for cataracts. The tips include an external elastomeric end cap having opposing proximal and distal end portions, the distal end portion having an aspiration port and a textured patch on an outer surface, the end cap sized and configured for polishing a capsular bag and/or contacting cortical fibers using the textured surface.

The textured patch can reside only on a distalmost end of the end cap.

The textured patch can cover only a rounded distal end of the end cap a distance forward of the aspiration portion.

The textured surface can be spaced apart between about 0.1 mm to about 5 mm from the aspiration port and other than the textured patch, the end cap has a smooth outer surface.

The distal end portion of the end cap can have a surface area and the textured patch surface occupies less than half the surface area.

The textured patch can occupy an elongate area of a sub-portion of the distal end portion of the end cap with the end cap having a non-textured smooth surface for at least a major portion of a surface area of the end cap.

Still other embodiments are directed to ophthalmic irrigation/aspiration devices. The devices include: an aspiration cannula, the cannula having a hub configured to attach to a handpiece and an open end opposite the hub; and a removable, external elastomeric tip adapted to enter a capsular bag of an eye of a patient, the tip sealing the open end of the cannula and characterized in that the tip comprises a distal end portion with an outer surface having a textured patch.

The textured patch can reside only on a distalmost end of the tip.

The textured patch can cover only a rounded distal end of the tip a distance forward of the aspiration portion.

The textured surface can be spaced apart between about 0.1 mm to about 5 mm from the aspiration port and other than the textured patch, the tip has a smooth outer surface.

The distal end portion of the tip can have a surface area, and wherein the textured patch surface occupies less than half the surface area.

The textured patch can occupy an area of a sub-portion of the distal end portion of the tip with the tip having a non-textured smooth surface for at least a major portion of a surface area of the tip.

The tip can include an end cap with a flange that is coupled to the cannula through a friction-fit between a portion of the end cap and the cannula, wherein, wherein the aspiration port is located at a distal tip of the end cap and the textured patch resides forward of the aspiration port on the tip.

The tip can include a sleeve that is external to the hub and hand piece, the sleeve further comprising a fluid irrigation channel and at least one associated port.

The textured surface can be spaced apart between about 0.1 mm to about 1 mm from a distal end of the tip.

The textured patch can occupy less than half a surface area of the distal end of the tip.

The textured patch can occupy an elongate narrow strip area of a sub portion of the distal end portion of the tip.

The end cap can be rubber.

The foregoing and other objects and aspects of the present invention are explained in detail in the specification set forth below.

It is noted that aspects of the invention described with respect to one embodiment, may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner These and other objects and/or aspects of the present invention are explained in detail in the specification set forth below.

### BRIEF DESCRIPTION OF THE DRAWINGS

FIG. 1 is a side schematic partial cutaway view of a device with an aspiration/irrigation tip according to embodiments of the present invention.

FIG. 2 is an end view of the device shown in FIG. 1.

FIG. 3 is a greatly enlarged view of the device shown in FIGS. 1 and 2.

FIG. 4 is a schematic illustration of a surgical aspiration tip suitable for cataract surgery according to embodiments of the present invention.

FIG. 5 is an end perspective view of another exemplary tip according to embodiments of the present invention.

FIG. 6 is an exploded view of the tip of FIG. 5.

FIG. 7 is a section view of the tip of FIG. 5.

FIG. 8 is an enlarged partial section view of another exemplary aspiration tip according to embodiments of the present invention.

FIG. 9 is an enlarged partial section view of a distal end portion of another tip configuration according to embodiments of the present invention.

FIGS. 10A-10E are partial cutaway views of examples of textured surfaces according to embodiments of the present invention.

FIG. 11 is an enlarged partial section view of a portion of a tip with a textured surface according to embodiments of the present invention.

FIG. 12 is an enlarged partial view of a device with an aspiration tip according to embodiments of the present invention.

FIGS. 13A and 13B are cross-sectional views taken along line 13-13 in FIG. 12 illustrating that the textured surfaces can be provided as a subset of a distal end, covering a portion of a perimeter (e.g., a circumference) of the sleeve/tip according to embodiments of the present invention.

FIG. 14A is an enlarged partial cutaway view of another embodiment of surgical tool with a tip suitable for cataract surgeries according to embodiments of the present invention.

FIG. 14B is a side cutaway view of the device shown in FIG. 14A, illustrating an open aspiration port according to embodiments of the present invention.

FIG. 14C is a side cutaway view of the device shown in FIG. 14A, illustrating a partially closed aspiration port according to embodiments of the present invention.

FIG. 15A is an enlarged partial cutaway view of another embodiment of surgical tool with a tip suitable for cataract surgeries according to embodiments of the present invention, illustrating a translating member and open aspiration port according to embodiments of the present invention.

FIG. 15B is an enlarged partial cutaway view of the tool shown in FIG. 15A illustrating the translating member and a partially closed aspiration port according to embodiments of the present invention.

FIG. 15C is a side cutaway view of the device shown in FIG. 15A, illustrating an open aspiration port according to embodiments of the present invention.

FIG. 15D is a side cutaway view of the device shown in FIG. 15A, illustrating a partially closed aspiration port according to embodiments of the present invention.

FIG. 16A is an enlarged partial cutaway view of another embodiment of surgical tool with a tip suitable for cataract surgeries according to embodiments of the present invention.

FIG. 16B is an enlarged partial cutaway view of the device shown in FIG. 16A, illustrating a sleeve rotated to partially occlude the aspiration port according to embodiments of the present invention.

FIG. 17 is a flow chart of exemplary operations that can be used to carry out embodiments of the present invention.

FIG. 18 is a flow chart of exemplary operations that can be used to carry out embodiments of the present invention.

### DESCRIPTION OF EMBODIMENTS OF THE INVENTION

The present invention will now be described more fully hereinafter with reference to the accompanying figures, in which embodiments of the invention are shown. This invention may, however, be embodied in many different forms and should not be construed as limited to the embodiments set forth herein. Like numbers refer to like elements throughout. In the figures, certain layers, components or features may be exaggerated for clarity, and broken lines illustrate optional features or operations unless specified otherwise. In addition, the sequence of operations (or steps) is not limited to the order presented in the figures and/or claims unless specifically indicated otherwise. In the drawings, the thickness of lines, layers, features, components and/or regions may be exaggerated for clarity and broken lines illustrate optional features or operations, unless specified otherwise.

The terminology used herein is for the purpose of describing particular embodiments only and is not intended to be limiting of the invention. As used herein, the singular forms, “a”, “an” and “the” are intended to include the plural forms as well, unless the context clearly indicates otherwise. It will be further understood that the terms “comprises,” “comprising,” “includes,” and/or “including” when used in this specification, specify the presence of stated features, regions, steps, operations, elements, and/or components, but do not preclude the presence or addition of one or more other features, regions, steps, operations, elements, components, and/or groups thereof.

It will be understood that when a feature, such as a layer, region or substrate, is referred to as being “on” another feature or element, it can be directly on the other feature or element or intervening features and/or elements may also be present. In contrast, when an element is referred to as being “directly on” another feature or element, there are no intervening elements present. It will also be understood that, when a feature or element is referred to as being “connected”, “attached” or “coupled” to another feature or element, it can be directly connected, attached or coupled to the other element or intervening elements may be present. In contrast, when a feature or element is referred to as being “directly connected”, “directly attached” or “directly coupled” to another element, there are no intervening elements present. Although described or shown with respect to one embodiment, the features so described or shown can apply to other embodiments.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein.

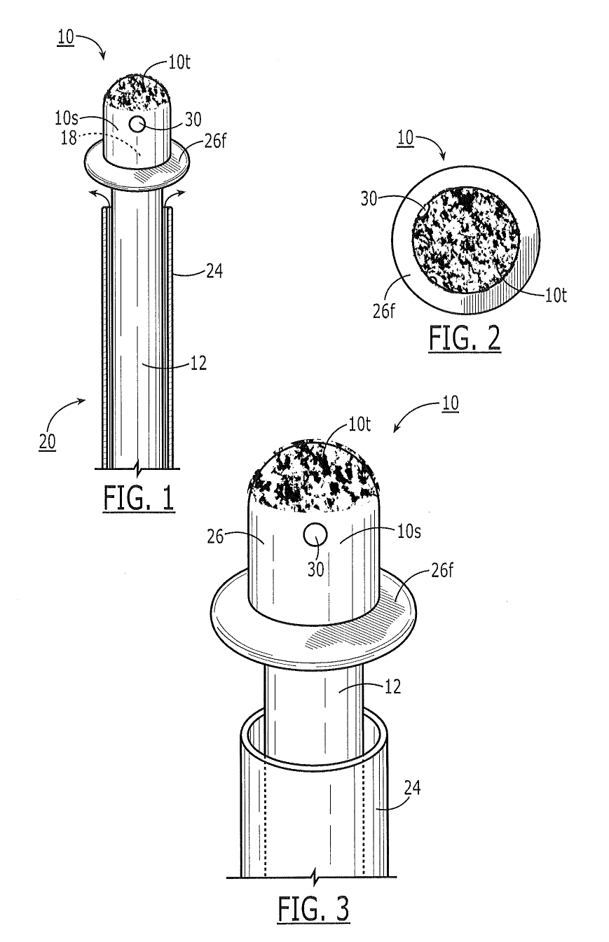

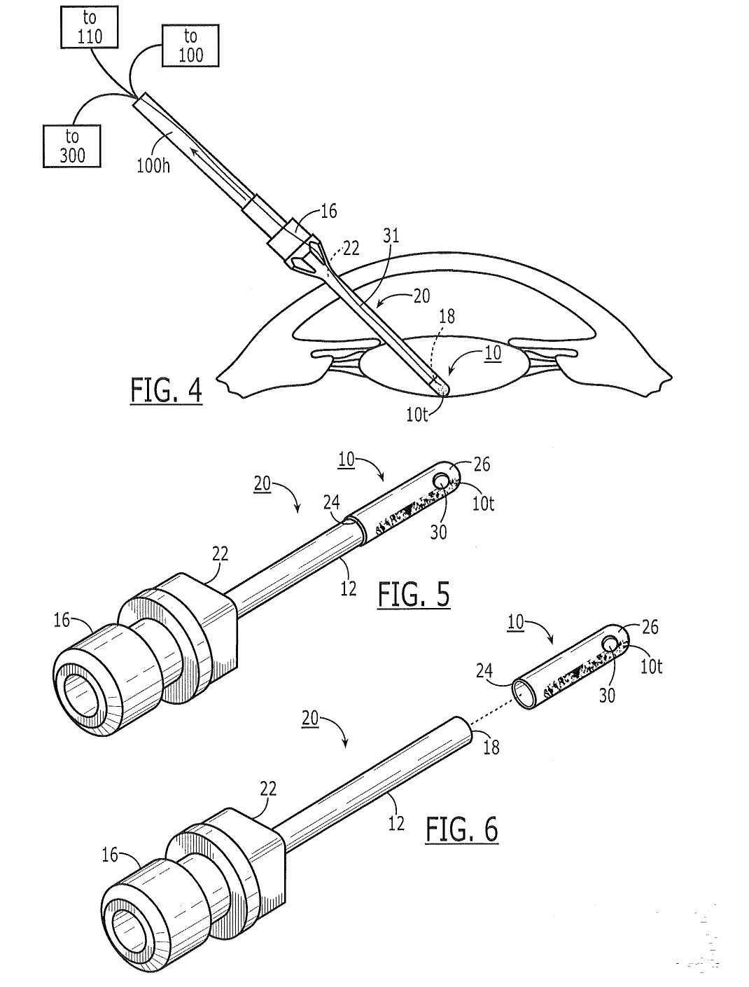

Referring now to the figures, FIG. 1 illustrates a surgical device 20 with a tip 10 having a textured surface 10t. Unlike the Micro Smooth® polymer tips from Alcon, Inc., the tip 10 which, in some embodiments, is configured to be placed in a capsule bag of an eye of a patient during surgery (FIG. 4), has a small sub-portion (“patch”) with a textured surface 10t.

The term “textured” refers to a surface that has a different surface finish or tactile surface pattern relative to smooth surfaces to provide a surface with increased grip and/or friction suitable for acting as an eraser on cortical fibers and/or for polishing the capsule bag. The textured surface can be substantially smooth but have increased friction or grip relative to non-textured (smooth) finish surfaces. The texture can be similar to the microetched portion on a Kratz capsule polisher such as the BD Visitec™ capsule polisher from Beaver-Visitec International.

The term “tip” refers to a distal end portion of a tool for cortical clean up and/or polishing of the capsule bag. The term “patch” refers to a small localized exterior textured surface region that is integral to the tip body, typically having a size that is less than 50% of a surface area of the tip body. Thus, the word “patch” refers to a size of the textured surface which can be formed directly into the surface of the tip body as will be discussed below and does not require, but can include, a separate element to provide the textured surface.

The word “about” means that the size or amount referred to can vary from the particular amount, typically by +/−10%.

The term “phacoemulsification” (also referred to as “phaco”) refers to both ultrasound and laser-based emulsification procedures used to disintegrate target interior eye tissue, typically the lens, for cataract surgery, as well as combinations of ultrasound and laser procedures. The term “electrical lead” refers to all electrical transmission paths including integrated conductive films, traces, filars, and cables.

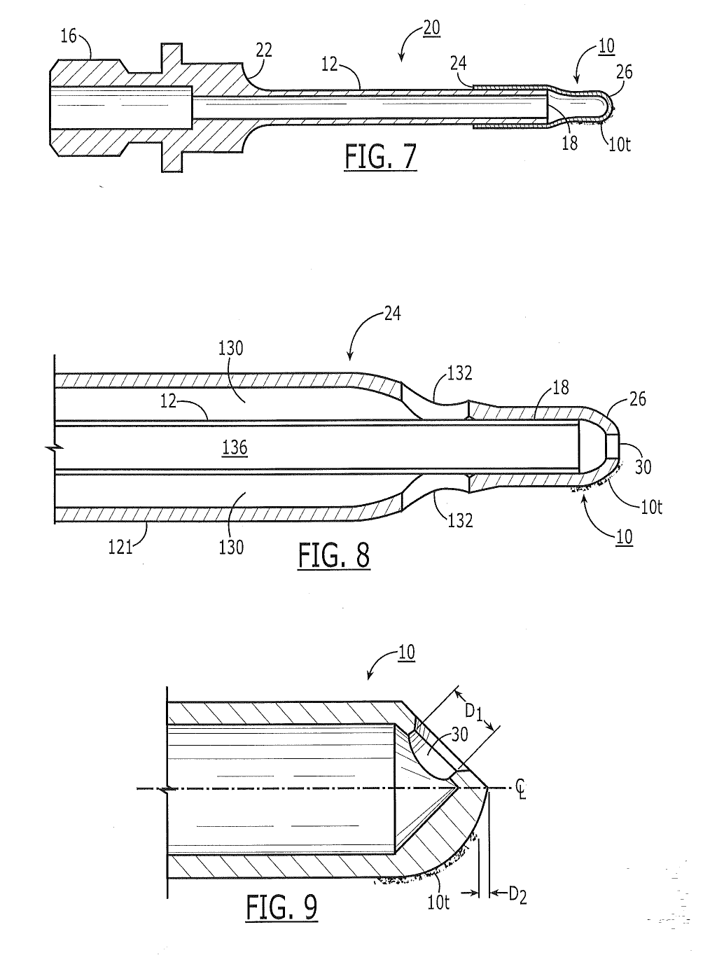

The textured surface 10t can be provided on a sub-portion of the exterior surface of a single-use (disposable) elastomeric end cap 26 as shown in FIGS. 1-9 that is attached to a an aspiration cannula 12 that defines the aspiration channel. The tip 10 can include at least one aspiration aperture 30 on an end portion thereof, typically a single aspiration aperture 30. The end cap 26 can comprise a monolithic material such as an elastomer or polymer including, but not limited to, silicone rubber.

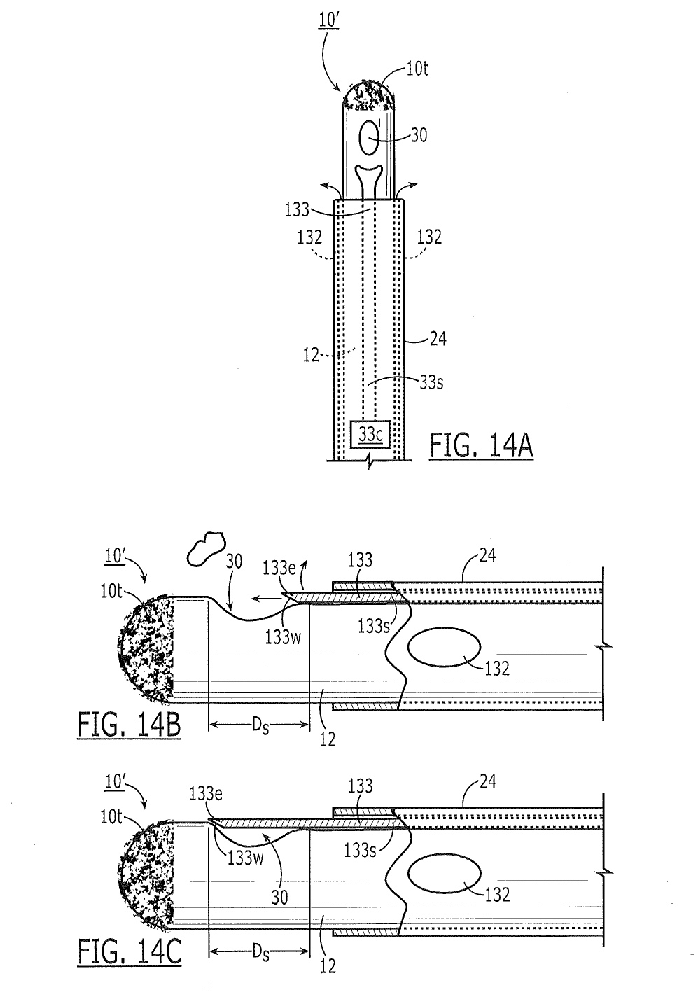

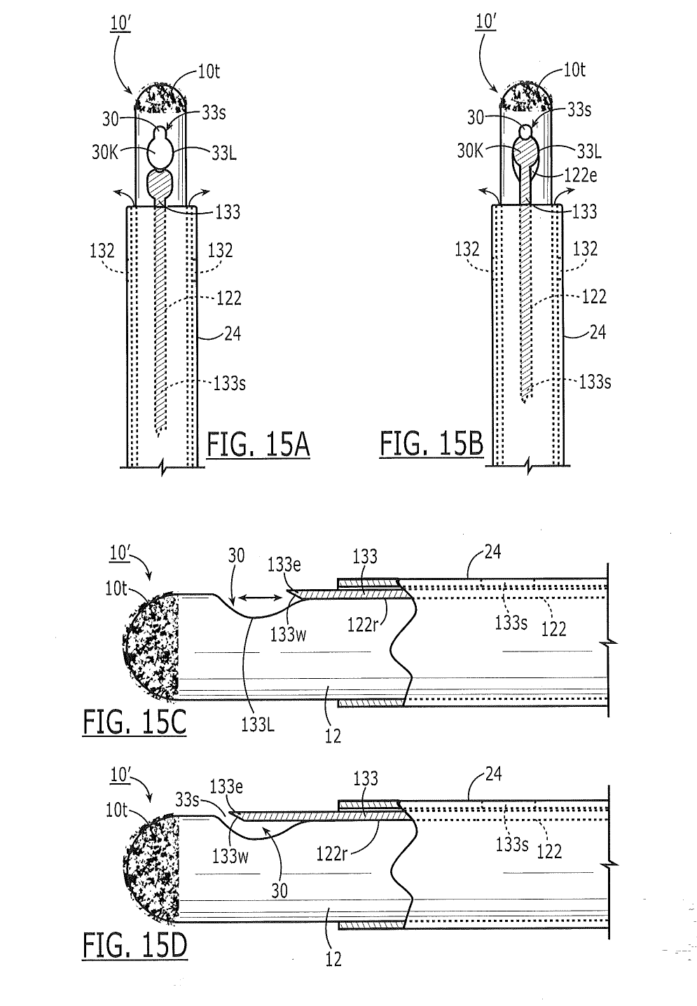

In some embodiments, the surgical device 20 with the multifunctional tip 10 can be used for ultrasound phacoemulsifcation procedures. In other embodiments, the tip 10 can be used for laser-phaco cataract procedures such as after or during femtosecond laser treatment to remove nuclear fragments and epinucleus. The tip 10 can provide irrigation and/or aspiration. In some embodiments, such as where used in lieu of ultrasound phaco, the tip 10 but may include a larger aspiration port 30 to accommodate the larger fragments (see, e.g., FIGS. 14A-C, FIGS. 15A-D).

FIGS. 1-3 show the device 20 as including an irrigation sleeve 24 that is separate from the end cap 26. In this embodiment, irrigant can flow between the cannula 12 and the sleeve 24. The end cap 26 can include a flange 26f that is rearward of the aspiration port 30 and the textured patch 10t. The cap 26 can have a smooth surface 10s about the aspiration port 30 and rearward thereof as shown in FIGS. 1-3, for example. The textured surface 10t can be spaced apart between about 0.1 mm to about 5 mm from the aspiration port 30 and other than the textured patch 10t, the tip 10 can have a smooth outer surface. The textured patch 10t can reside only on the tapered distal most end of the tip or end cap so that the texture terminates proximate a junction that merges into the smooth vertical outerwalls. This textured patch 10t can occupy a small length of the distal end of the tip 10 and/or end cap 26, similar to an eraser on a pencil. This small length can be between about 0.1 mm to about 3 mm.

FIGS. 5-7 show a sleeve 24 that attaches to an outer surface of the cannula 12 without leaving an annular space for irrigant. FIG. 5 shows that the end cap 26 can be integral to the sleeve 24. FIG. 8 also shows that the end cap 26 can be integral to the sleeve 24 with the sleeve 24 configured to define a flow channel 130 and irrigation port(s) 132 and attach to the aspiration cannula 12. The tip 10 and/or end cap 25 can have a very small width, such as less than about 2 mm, including about 0.9 mm and about 1.1 mm, for example.

Referring to FIG. 4, the tool 20 can releasably connect, via a hub 16, to an aspiration 100 and/or irrigation system 110 with a handpiece 100h as is well known to those of skill in the art. The cannula 12 can be open at distal end 18 and can be attached to hub 16 at proximal end 22. The tool 20 may also optionally be in communication with an ultrasound source 300 and may include an electrical lead 31 that extends to the tip 10.

As shown in FIGS. 1-3, the textured surface 10t can cover only the distal end of the tip 10 and typically terminates prior to the aspiration port(s) 30 so that the remainder of the tip 10, end cap 26 or sleeve 24 is smooth 10s.

In some embodiments, the textured surface 10t can reside on an opposite surface from an irrigation/aspiration port 30 (FIGS. 9, 13A, 13B). The textured patch region 10t can have a length “L” that is recessed axially inward a distance from the distal tip end and that terminates away from the proximal end of the tip that is attached to the tool body (FIGS. 5, 9).

In the embodiment shown in FIG. 8, the distal end 18 of cannula 12 is sealed by a reduced diameter portion of the sleeve 24, which is typically integrally formed at the distal end of shaft 121. The sleeve 24 is generally tubular in shape and closed on its distal end except for aperture 20. The reduced diameter of the sleeve 24 is configured to seal tightly about the distal end portion 18 of cannula 12. The sleeve 24 is also configured to form a coaxial gap 130 around cannula 12. Gap 130 allows irrigation flow down gap 130 and out ports 132. When vacuum is applied to interior lumen 136 of cannula 12, material can be aspirated through port 30, down interior lumen 136 and out of tool 20.

FIGS. 1-3, 5 and 9 show the aperture 30 spaced away from the distal end of the tip 10 while FIG. 8 shows that the aperture 30 can reside on the distal end of the tip 26.

FIG. 9 shows the aperture 30 on an end of the tip but offset from a centerline of the end cap 26 and/or tip 10. FIG. 9 also shows the textured surface 10t on a rounded portion to terminate prior to an edge adjacent a planar surface holding the aperture 30. The aperture 30 can have a size D1.

The textured surface 10t can be configured to occupy or reside on less than the entire end of the tip. The textured surface 10t can be recessed or offset a distance from the distal end of the tip, such as a distance “D2” as shown, for example, in FIG. 9. The distance “D2” can be between about 0.1 mm to about 10 mm, typically between about 1 mm to about 3 mm.

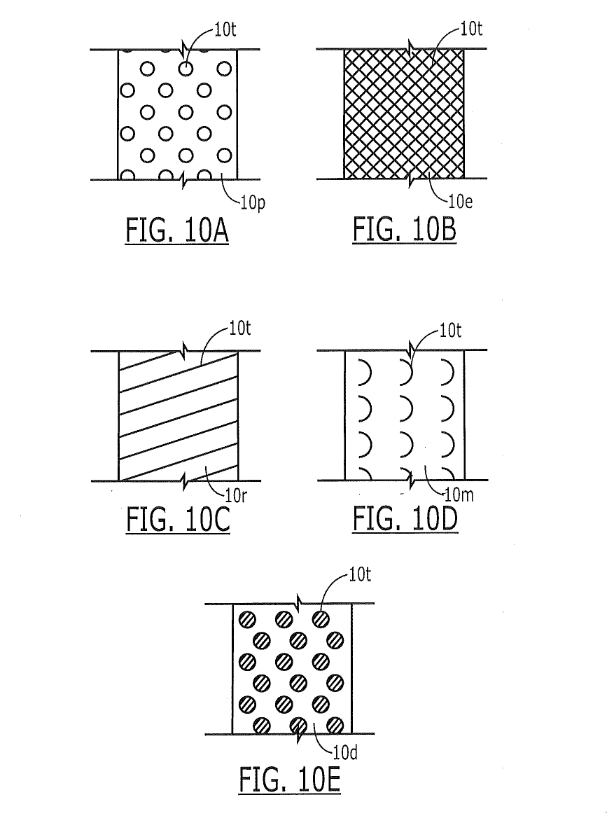

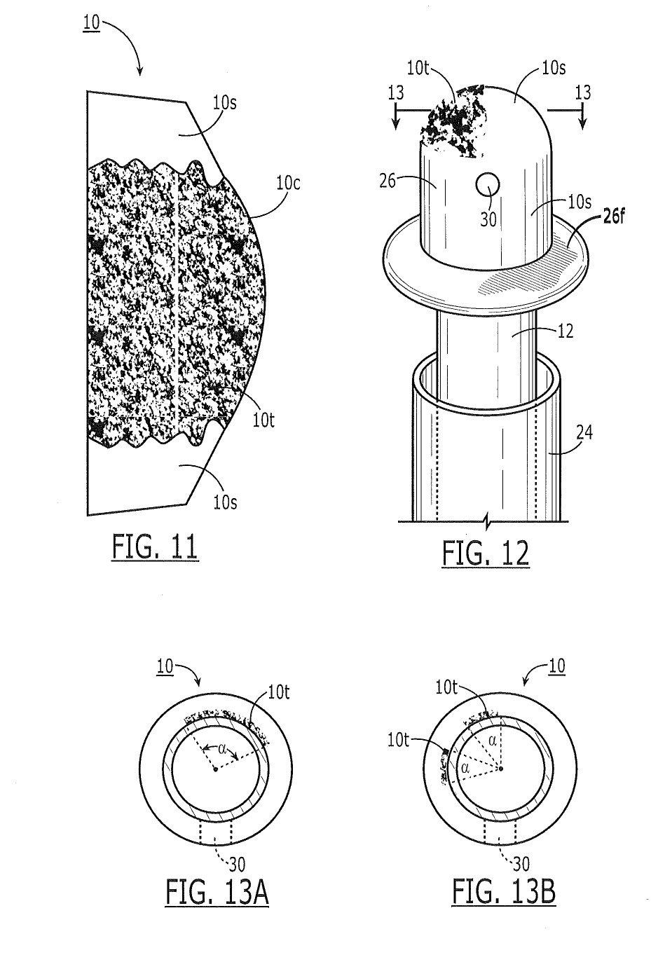

FIGS. 10A-10E and 11 illustrate examples of different textured surface 10t configurations. FIG. 10A illustrates the textured surface 10t includes particulates such as nanoparticles or granular material as a surface coating. FIG. 10B illustrates an embossed surface 10e. The embossed surface can be in a regularly or irregularly repeating fashion of one or more defined shapes. Although shown as a generally diamond shaped pattern, other shapes may be used including honeycomb, polygons, circles, or other shapes. FIG. 10C illustrates a slightly roughened surface 10r. FIG. 10C illustrates the textured surface can include mounds while FIG. 10D illustrates dimples. Combinations of projections and recessions, e.g., mounds and dimples, can also be used. FIG. 11 illustrates a cluster 10c of irregular features on an exterior surface of the tip 10. FIG. 11 also illustrates that the tip can include a smooth surface 10s adjacent the textured surface 10t. Combinations of the above or other textures or patterns may be used.

FIG. 10 illustrates that the tip 10 can include the textured surface 10t on an elongate segment of the sleeve 24 which can be on one side of the tip 10, typically terminating proximate to the port 30 but can extend a distance rearward from the port 30, such as between about 0.1 mm to about 5 mm, and in some embodiments between about 1 mm to about 5 mm.

In some embodiments, the textured surface 10t can reside over a sub-portion portion of a perimeter region of the tip 10 as shown in FIGS. 12, 13A and 13B. FIGS. 13A and 13B illustrate that the textured surface 10t can reside or extend over less than about half the perimeter (which may be a circumference) of the tip 10. The textured surface 10 can be provided as a narrow patch segment on the tip 10, such as between about 10% to about 30% of the circumference or other perimeter shape. FIG. 12 illustrates that the textured patch can reside over less than an entire distal end surface, typically so as to cover less than about 50% thereof. FIGS. 13A and 13B are exemplary section illustrations of the tip 10 taken along lines 13-13 in FIG. 12. FIGS. 13A and 13B illustrate that the textured surface 10t can reside over less than about 90 degrees of the circumference of the tip 10. FIG. 13B illustrates that the textured surface 10t can be discontinuous about the perimeter. The textured surface 10t can occupy less than half a surface area of the perimeter of the distal end portion of the tip 10 or sleeve. In some embodiments, the textured surface 10t can be provided as a narrow strip or patch on the distal end portion of the tip. In some embodiments, the textured surface 10t can reside about a defined angle “α” that is typically less than 180 degrees, such as between about 15 degrees to about 120 degrees, such as about 30 degrees, about 45 degrees, about 60 degrees, about 75 degrees, about 90 degrees, about 110 degrees, for example. FIG. 13B illustrates two spaced apart textured segments 10t can be used although more than two, such as between 2-10 segments or more can be used. The segments can have the same or different angular coverage “α”.

Some of the textured surface patch configurations may allow a surgeon to rotate the tip 10 to enter the capsule so that the tip 10 contacts the capsule with a smooth surface during insertion (or retraction). The surgeon can then rotate the tip 10 to erase cortical fibers and/or polish the capsule and/or remove nuclear fragments using the textured surface 10t. The partial textured surface 10t can be provided with a color contrast to other portions of the tip to allow for ease of viewing during a surgical procedure.

The textured surface 10t of the tip can be formed or provided in any suitable manner. For example, coating the sleeve using a biocompatible coating, such as a coating with particulates, dipping the sleeve in an acid rinse or ultrasonic bath (for pitting), embossing the sleeve, or molding the sleeve in a mold which provides the desired surface texture or attaching a small separate patch material onto a portion of the outer surface of the tip. In some embodiments, the textured surface of the sleeve 24 can be rough but without jagged edges that might tear the capsule bag. The roughened textured surface 10t may be formed in any suitable manner such as sandblasting, pinging, rubbing against a rough tool or sand paper and the like.

FIGS. 14A-C illustrate another embodiment of the surgical device 10′. The device 10′ can include the textured tip 10t as described above for polishing and also or alternatively can include a cortex/lens removal system with a longitudinally translatable member 133 that can move between distal and proximal directions. In operation, lens fragments, especially nucleus or cortex fragments try to be aspirated via port 30, but larger ones can get stuck. The translating member 133 can be configured to have an oscillating and/or reciprocating movement to “chop”, fragment, crush or otherwise reduce the larger lens fragments in size (those that are trying to be aspirated via the port 30 but are too large) with the forward/aft oscillating and/or reciprocal movement of the translating member 133. To be clear, although shown with a textured tip 10t, in some embodiments, the device 10′ can be configured without the textured tip 10t.

The stroke “Ds” of the translating member 133 during the oscillation/reciprocal movement can be very short, e.g., the forwardmost position can terminate proximate the leading end of the aspiration port 30. The stroke distance can be limited and controlled and can be between about 2 mm to about 0.1 mm, typically between about 2 mm to about 0.5 mm. The stroke cycle can be rapid or slow, typically between 1-10 Hz. The cycle speed and distance may be adjustable or selectable from a predefined operational list that programmatically controls the movement upon activation of a control 33c by a user. The control 33c can comprise a user-actuated control in communication with the shaft 133s, such as manual control of any suitable type, including, for example, a switch, button, thumbwheel, foot pedal or may comprise an electronic control such as a voice activated control.

The user-actuation control 33c is configured to control the reciprocating movement and/or oscillation of the translating member 133, e.g., a finger press on the shaft or foot pedal position. The control 33c can allow open/oscillating/closed, just oscillating, or oscillating and closed action of the member 133. Separate controls may also be used for the different actions.

The aspiration port 30 can be sized to be able to engulf lens fragments. The ones that fit in the port 30 can simply be aspirated, but the larger ones that get stuck can then be “chopped”, fragmented or otherwise reduced in size with the extension and/or reciprocal movement of the translating member 133. The port 30 can have a size that is about 1-3 mm in diameter. Non-circular irrigation port shapes may also be used and the port 30 can have a width and length that is between 1-3 mm.

As shown, the sleeve 24 can also have at least one irrigation port 132, typically two ports, one on each lateral side of the translating member 133.

The leading edge of the translating member 133e can have a wedge configuration to trap lens fragments. As shown, the wedge 133w can angle down with a longer end being above a lower shorter end.

The shaft 133s can slidably reside in a correspondingly shaped (mating) groove 122 in the outer wall of the cannula 12 (or inner wall of the sleeve 24) for alignment and orientation control (e.g., similar to a “tongue and groove” or rail configuration). The groove 122 can extend down the center of the device 10′. The translating member 133 can be extended when the fragmenting is complete to aspirate via a small gap space left between the end of the translating member 133e and the underlying partially closed aspiration port 30 (FIG. 14C). The tip 10t can then be used to polish the capsule. The device can be configured to complete the procedure after the laser has done its part in dismantling the lens. Preferably, no ultrasound is required for the procedure (a safe and cost effective solution to avoid ultrasound).

FIGS. 15A-15D illustrate a similar configuration as the embodiment shown with respect to FIGS. 14A-14C. In this embodiment, the aspiration port 30 can have two segments, a “large” size segment 33L and a small size segment 33s. Like before, the translating member 133 can reside in a groove 122 (e.g., channel or recess) on an outer surface of the cannula 12 or on an inner surface of the sleeve 24 (or combinations thereof). The groove 122 can extend down the center of the device 10′. The translating member 133 can translate forward and aft to perform the lens crushing and, when desired, close the larger port 33L.

The aspiration port 30 can have an irregular shape such as a “keyhole” shape 30k (FIG. 15A) with the larger and smaller segments 33L, 33s. In other embodiments, two separate adjacent ports can be provided, one smaller than another (not shown). The smaller port or port segment 33s can have a diameter of about 0.25 mm to about 0.5 mm. The larger port or port segment 33L can be 2 times to ten times larger than the smaller port or port segment 33s.

The leading end 133e of the translating member can have a shape that substantially corresponds to a shape of the larger segment of the port 33L so as to occlude the underlying portion of the port 30, e.g., larger segment 33L. As shown, the leading end 133e has a circular shape with a tapered or wedge shaped end that can trap lens fragments over the port 30. The leading end of the groove 122 can have a correspondingly shaped, closed surface recess 122e. As shown, the recess 122e is wider than the long recess of the shaft 122s and terminates proximate the port 30.

The larger section or larger port 33L can reside a further distance away from the distal tip of the device 10t relative to the small portion 33s. The larger segment of the port or larger port 33L is sized to be able to engulf lens fragments. The lens fragments that fit in port 33L can simply be aspirated, but the larger ones that get stuck can then be “chopped”, fragmented, crushed or otherwise reduced in size with the extension and/or reciprocal movement of the translating member 133.

FIGS. 15C and 15D illustrate, in side view, the movement of the translating member 133, e.g., forward and aft movement, to fragment, crush or otherwise act on the lens fragments, when needed. Once the lens is fully removed, the translating member 133 can be positioned to cover the larger port 33L, e.g., the bottom of the keyhole 30k, leaving the smaller port 33s (e.g., a top of the keyhole) to aspirate the cortex safely. The tip 10t can then be used to polish the capsule. This should be all that is required after the laser has done its part in dismantling the lens. Preferably, no ultrasound is;required for the procedure (a safe and cost effective solution to avoid ultrasound).

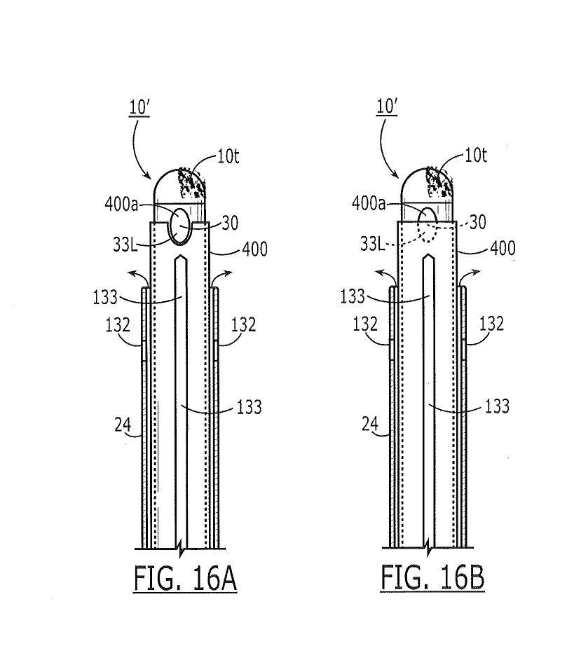

FIGS. 16A and 16B illustrate an alternate embodiment where the device 10′ can include a thin rotatable sleeve 400 that resides over the cannula 12 and under the translating member 133 and has a wall with a large aperture 40a allowing exposure of the port 30 and at least one laterally spaced apart small aperture 400s. When rotated so that the small aperture 400s overlays the port 30, the sleeve 400 can partially close the port 30 and provide the small access port segment 33s. Thus, in this embodiment, the translating member 133 is not required to close against the port 30 to form the small port 33s as the sleeve 400 cooperates with the tip 10 to aspirate fragmented lens after the oscillation/reciprocal action of the leading end of the translating member 133. Again, the device 10′ can include a user-actuated control 33c that is in communication with the shaft 12s for controlling the reciprocating movement or oscillation of the translating member 133,

The device 10 may be particularly suitable for laser-phaco. In the past, sometimes when a small nuclear piece is left behind and noticed during cortex removal, a second instrument is used to smash it into the tip while aspirating. The multi-functional tool 10′ with the multi-functional tip 10 having the textured external surface 10t can avoid the need for such a second device and/or ultrasound phaco.

Examples of currently available femtosecond laser optical systems are believed to include Alcon LenSx (Alcon Laboratories, Ft Worth, Tex., USA), OptiMedica Catalys (Optimedica Corp, Calif., USA), LensAR(LensAR Inc, Fla., USA) and Technolas (Technolas Perfect Vision GmbH, Germany). The laser systems typically include an anterior segment imaging system, patient interface and femtosecond laser to image, calculate and deliver the laser pulses. In some embodiments, the surgical tool 10 with the multifunctional tip can be used after or during femtosecond laser surgery to remove nuclear fragments and epinucleus. The textured tip 10t can provide irrigation and/or aspiration port(s) 30 sized and configured to accommodate the larger fragments typically generated by this procedure. The tip 10 can be in communication with an aspiration source (e.g., vacuum) and optionally an ultrasound source 300.

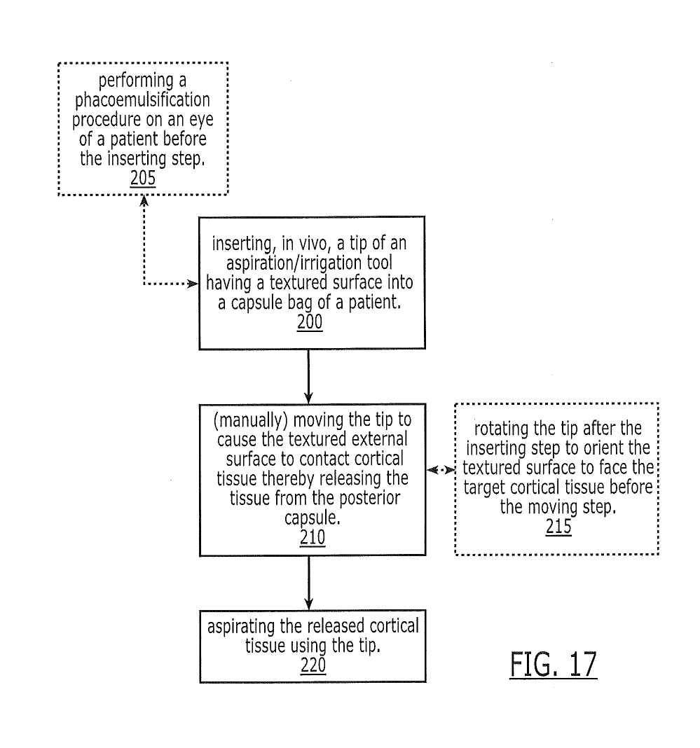

FIG. 17 illustrates operations that can be used to carry out embodiments of the present invention. As shown, a tip of an aspiration/irrigation tool having a textured surface can be inserted, in vivo, into a posterior capsule bag of a patient (block 200). The tip can be (typically manually) moved to cause the textured surface to contact cortical tissue thereby releasing the tissue from the posterior capsule (block 210). The released cortical tissue can be aspirated using the tip (block 220).

The inserting can be carried out after a phacoemulsification procedure is performed on an eye of a patient (block 205).

In some embodiments, the tip can optionally be rotated after the inserting step to orient the textured surface to face the target cortical tissue before the moving step (block 215).

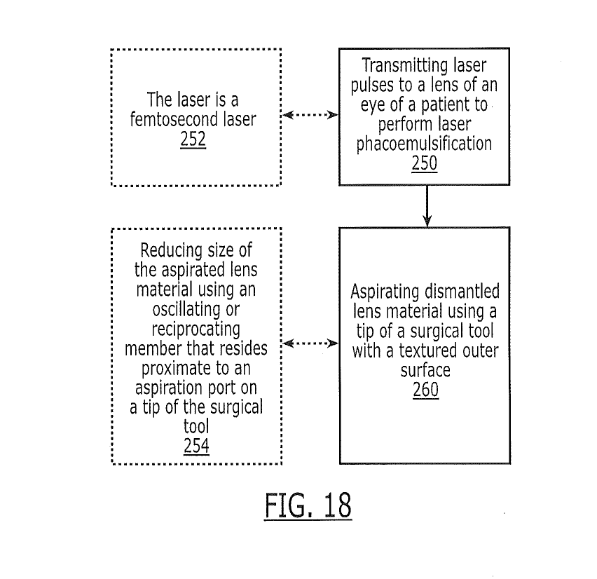

FIG. 18 illustrates exemplary operations of an alternate embodiment of the present invention. In this cataract surgery, a femtosecond laser procedure can be used. The method can include transmitting laser pulses to a lens of an eye of a patient to perform laser phacoemulsification (block 250). For example, a series of defined laser pulses can be transmitted to a lens of a patient's eye to dismantle the lens. The laser can be a femtosecond laser (block 252) for laser emulsification (“laser phaco”) which may eliminate the requirement for ultrasound phaco. The laser may use a modified LASIK laser that is configured to allow for bladeless cataract surgery, such as lasers employing a disk that allows for LASIK systems to be used for cataract surgeries such as the Newsom Bladeless Laser Disk™. In any event, the method includes aspirating dismantled lens material using a tip of a surgical tool with a textured outer surface (block 260). The method can include using an oscillating and/or reciprocating member to reduce size of larger lens material prior to suctioning out of an aspiration port (block 254).

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. In the claims, means-plus-function clauses, if used, are intended to cover the structures described herein as performing the recited function and not only structural equivalents but also equivalent structures. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

That which is claimed is:

1. A method of performing cataract surgery, comprising:
performing a phacoemulsification procedure on an eye of a patient; then
inserting, in vivo, an elastomeric tip of an aspiration/irrigation tool having an aspiration port and a textured patch on an outer surface thereof into a capsule bag of a patient; then
moving the tip to cause the textured patch to contact cortical tissue thereby releasing it from a posterior capsule; and then
aspirating the released cortical tissue using an aspiration port on the tip.

2. The method of claim 1, wherein the tip has a non-textured smooth outer surface proximate the irrigation/aspiration port, the smooth outer surface covering a greater surface area than a surface area of the textured patch.

3. The method of claim 1, wherein the textured patch resides only on a distalmost end of the tip.

4. The method of claim 1, wherein the textured patch covers only a rounded distal end of the tip a distance forward of the aspiration portion.

5. The method of claim 2, wherein the tip has a smooth surface opposite the textured patch, the method comprising rotating the tip so that the textured surface faces the cortical tissue after the inserting step.

6. The method of claim 1, further comprising oscillating and/or reciprocating a translating member proximate the aspiration port to crush, fragment, or otherwise reduce size of lens pieces in the eye of the patient before the moving the tip step to remove loose lens material after the phacoemulsification.

7. The method of claim 1, wherein the performing step comprises transmitting laser pulses to a lens in the eye of the patient to perform the phacoemulsification.

8. The method of claim 1, wherein the translating is carried out to move a leading end of a translating member aft and forward a distance between 0.5 mm and 2 mm to crush, fragment, chop or otherwise reduce the size of the loose lens material proximate the aspiration port.

9. The method of claim 1, further comprising reducing an intake size of the aspiration port using the translating member after the oscillating or reciprocating movement to safely remove cortex lens material.

10. A multi-purpose irrigation/aspiration tip for use in combination with a surgical system for cataracts, comprising:
an external elastomeric end cap having opposing proximal and distal end portions, the distal end portion having an aspiration port and a textured patch on an outer surface, the end cap sized and configured for polishing a capsular bag and/or contacting cortical fibers using the textured surface.

11. The tip of claim 10, wherein the textured patch resides only on a distalmost end of the end cap.

12. The tip of claim 10, wherein the textured patch covers only a rounded distal end of the end cap a distance forward of the aspiration portion, and wherein the end cap is rubber and sterilized for surgical use.

13. The tip of claim 10, wherein the textured surface is spaced apart between about 0.1 mm to about 5 mm from the aspiration port and other than the textured patch, the end cap has a smooth outer surface.

14. The tip of claim 10, wherein the distal end portion of the end cap has a surface area, and wherein the textured patch surface occupies less than half the surface area.

15. The tip of claim 10, wherein the textured patch occupies an elongate area of a sub-portion of the distal end portion of the end cap with the end cap having a non-textured smooth surface for at least a major portion of a surface area of the end cap.

16. The tip of claim 10, further comprising an aspiration port and a translating member with a leading edge, the translating member in communication with a user control, wherein the translating member leading edge is configured to selectively oscillate or reciprocate a short distance over an external surface of the tip proximate the aspiration port.

17. The tip of claim 10, wherein the aspiration port has a keyhole shape with a small portion merging into a larger portion, and wherein the translating member has a leading end with a shape corresponding to a shape of the larger portion.

18. An ophthalmic irrigation/aspiration device, comprising:
an aspiration cannula, the cannula having a hub configured to attach to a handpiece and an open end opposite the hub; and
a removable, external elastomeric tip adapted to enter a capsular bag of an eye of a patient, the tip sealing the open end of the aspiration cannula and characterized in that the tip comprises a distal end portion with an outer surface having a textured patch and an aspiration port.

19. The device of claim 18, wherein the textured patch resides only on a distalmost end of the tip.

20. The device of claim 18, wherein the textured patch covers only a rounded distal end of the tip a distance forward of the aspiration portion.

21. The device of claim 18, wherein the textured surface is spaced apart between about 0.1 mm to about 5 mm from the aspiration port and other than the textured patch, the tip has a smooth outer surface.

22. The device of claim 18, wherein the distal end portion of the tip has a surface area, and wherein the textured patch surface occupies less than half the surface area.

23. The device of claim 18, wherein the textured patch occupies an area of a sub-portion of the distal end portion of the tip with the tip having a non-textured smooth surface for at least a major portion of a surface area of the tip.

24. The device of claim 18, wherein the tip comprises an end cap with a flange that is coupled to the cannula through a friction-fit between a portion of the end cap and the cannula, wherein, wherein the aspiration port is located at a distal tip of the end cap and the textured patch resides forward of the aspiration port on the tip.

25. The device of claim 18, wherein the tip comprises a sleeve that is external to the hub and hand piece, the sleeve further comprising a fluid irrigation channel and at least one associated port.

26. The device of claim 18, wherein the textured surface is spaced apart between about 0.1 mm to about 1 mm from a distal end of the tip.

27. The device of claim 18, wherein the textured patch occupies less than half a surface area of the distal end of the tip.

28. The device of claim 18, wherein the textured patch occupies an elongate narrow strip area of a sub portion of the distal end portion of the tip.

29. The device of claim 18, wherein the end cap is rubber.

30. The device of claim 18, further comprising a longitudinally translating member with a leading end configured to oscillate and/or reciprocate over an external surface of the tip proximate the aspiration port to reduce a size of loose lens material in an eye of a patient.

31. The device of claim 18, wherein the aspiration port has a keyhole shape with a small portion merging into a larger portion, and wherein the translating member has a leading end with a shape corresponding to a shape of the larger portion.

32. The device of claim 19, wherein the longitudinally translating member slidably resides in a groove extending in an outerwall of the aspiration cannula, and wherein the device further comprises a user actuation control in communication with a shaft of the translating member to cause the translating member to oscillate and/or reciprocate.

33. The device of claim 32, wherein the translating member has a wedge shaped leading end.

34. The device of claim 31, wherein the leading end of the translating member has a substantially circular shape that is sized and configured to overlie and occlude a portion of the aspiration port so that the smaller segment defines an active part of the aspiration port.

35. An ophthalmic irrigation/aspiration device, comprising:
an aspiration cannula, the cannula having a hub configured to attach to a handpiece and an open end opposite the hub;
a removable, external elastomeric tip adapted to enter a capsular bag of an eye of a patient, the tip sealing the open end of the aspiration cannula, wherein the tip comprises a distal end portion with an aspiration port; and
a longitudinally translating member held over the cannula configured to slidably travel over the cannula, the longitudinally extending member having a leading end configured to oscillate and/or reciprocate over an external surface of the tip proximate the aspiration port to reduce a size of loose lens material in an eye of a patient.

36. The device of claim 35, wherein the aspiration port has a keyhole shape with a small portion merging into a larger portion, and wherein the translating member has a leading end with a shape corresponding to a shape of the larger portion.

37. The device of claim 35, wherein the longitudinally translating member slidably resides in a groove extending in an outerwall of the aspiration cannula, and wherein the device further comprises a user actuation control in communication with a shaft of the translating member to cause the translating member to oscillate and/or reciprocate.

38. The device of claim 35, wherein the translating member has a wedge shaped leading end.

39. The device of claim 35, wherein the leading end of the translating member has a substantially circular shape that is sized and configured to overlie and occlude a portion of the aspiration port so that the smaller segment defines an active part of the aspiration port

40. A method of performing cataract surgery, comprising:
performing a phacoemulsification procedure on an eye of a patient;
inserting, in vivo, an elastomeric tip of an aspiration/irrigation tool having an aspiration port into a capsule bag of a patient;
moving a leading edge of a translating member back and forth a short distance over an external surface of the tip proximate the aspiration port to reduce size of lens fragments; then
aspirating the lens fragments through the aspiration port on the tip.

41. The method of claim 40, wherein the aspiration port has a first segment with a smaller size than a second segment, and wherein the leading edge of the translating member resides over the second segment during the aspiration.
